(19) United States
(12) Patent Application Publication
(10) Pub. No.: US 2026/0092907 A1
(43) Pub. Date: Apr. 2, 2026

(54) TESTING DEVICE FOR GAS SENSOR

(71) Applicant: Ningbo University, Ningbo City (CN)

(72) Inventors: Jie Zou, Ningbo City (CN); Jingyuan Sun, Ningbo City (CN); Jiawen Jian, Ningbo City (CN); Xiaoqing Jiang, Ningbo City (CN); Changkun Zhu, Ningbo City (CN); Jie Wang, Ningbo City (CN)

(21) Appl. No.: 18/901,078

(22) Filed: Sep. 30, 2024

(51) Int. Cl. G01N 33/00

(52) U.S. Cl. CPC G01N 33/007

(57) ABSTRACT

A testing device for a gas sensor is provided. The testing device for a gas sensor includes a thermostatic bath and a gas testing chamber arranged above the thermostatic bath. A humidity tank for adjusting gas humidity and a sample tank for testing the gas sensor are provided in the gas testing chamber. The sample tank is communicated with the humidity tank through a gas circuit, and the humidity tank and the sample tank extend into the thermostatic bath, so that the temperature of the humidity tank is consistent with that of the sample tank. The humidity tank and the sample tank extend into the thermostatic bath, the thermostatic bath is configured for ensuring that the temperature of the humidity tank is consistent with that of the sample tank.

### TECHNICAL FIELD

The present disclosure relates to the technical field of sensor testing, in particular to a testing device for a gas sensor.

### BACKGROUND

The temperature characteristics and humidity characteristics of the gas sensor need to be controlled by applicable testing devices. Therefore, it is of great significance to make a testing device suitable for the prepared sensor for the preliminary test of the sensor. In the existing testing device, the humidity generator unit and the sample tank are arranged separately. When tested in a low-temperature environment, gas with a specific humidity value adjusted by the humidity generator unit enters into the sample tank. However, if the temperature of the gas with the adjusted humidity value is higher than that in the sample tank, the gas can be condensed on the surface of the sample tank and the gas circuit for conveying the gas, and then the humidity of the gas is reduced, which affects the testing precision of the gas sensor.

Therefore, at present, those skilled in the art urgently need to solve the technical problem that how to design a testing device for a gas sensor to prevent the humid gas from being condensed in the process of entering into the sample tank, so that the accuracy of gas humidity is ensured to improve the testing precision of the gas sensor.

### SUMMARY

In order to solve the above-mentioned problem, the present disclosure provides a testing device for a gas sensor so as to prevent humid gas from being condensed in the process of entering into a sample tank, and then the accuracy of gas humidity is ensured to improve the testing precision of the gas sensor.

In order to achieve the-mentioned purpose, the present disclosure provides the following scheme. A testing device for a gas sensor includes a thermostatic bath and a gas testing chamber arranged above the thermostatic bath, where a humidity tank for adjusting gas humidity and a sample tank for testing the gas sensor are provided in the gas testing chamber, the sample tank is communicated with the humidity tank through a gas circuit, and the humidity tank and the sample tank extend into the thermostatic bath, so that a temperature of the humidity tank is consistent with that of the sample tank.

Compared with the prior art, some embodiments have the following technical effects.

The humidity tank and the sample tank extend into the thermostatic bath, and the thermostatic bath is configured for ensuring the temperature consistency of the humidity tank and the sample tank. In the process that the humid gas enters into the sample tank, temperature difference does not exist in the gas circuit, so the humid gas cannot be condensed, and then the accuracy of gas humidity is ensured to improve the testing precision of the sensor.

### BRIEF DESCRIPTION OF THE DRAWINGS

FIG. 1 is an explosive view of a testing device for a gas sensor disclosed in the embodiment of the present disclosure.

FIG. 2 is a structural schematic diagram of a thermostatic bath in a testing device for a gas sensor disclosed in the embodiment of the present disclosure.

FIG. 3 is a structural schematic diagram of a gas testing chamber in a testing device for a gas sensor disclosed in the embodiment of the present disclosure.

FIG. 4 is an integral structural schematic diagram of a testing device for a gas sensor disclosed in the embodiment of the present disclosure.

Reference signs: 1 thermostatic bath; 2 liquid inlet; 3 gas testing chamber; 4 humidity tank; 5 first air inlet passage; 6 humidity tank cover plate; 7 connecting clamping sleeve; 8 circuit board; 9 probe structure; 10 second air inlet passage; 11 sample tank; 12 groove; 13 air outlet passage; 14 liquid outlet; 15 seal ring; 16 pagoda-shape connector; 17 first air inlet port; 18 second air outlet port; and 19 seal ring groove.

### DETAILED DESCRIPTION OF THE EMBODIMENTS

The present disclosure aims to provide a testing device for a gas sensor so as to prevent humid gas from being condensed in the process of entering into a sample tank, and then the accuracy of gas humidity is ensured to improve the testing precision of the gas sensor.

The present invention is further described in detail in conjunction with the following attached figures and specific embodiments.

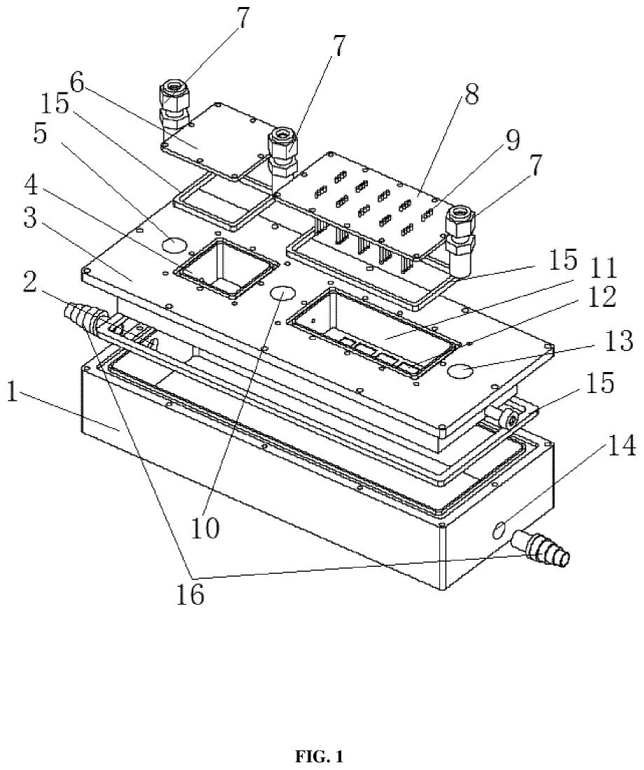

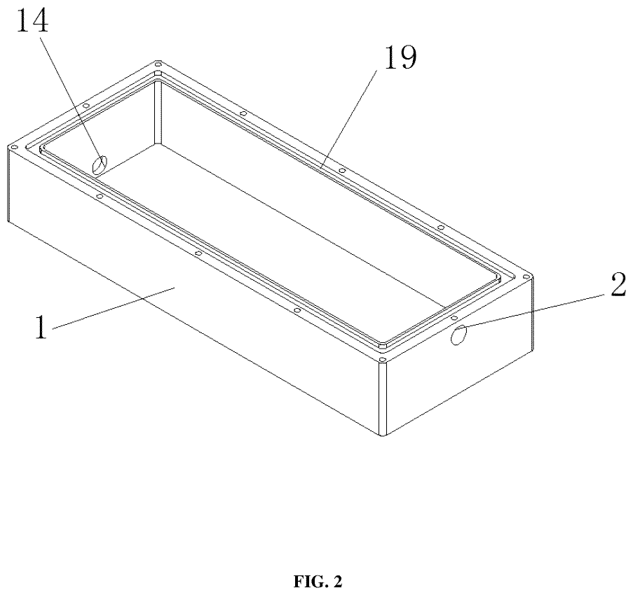

Referring to FIG. 1 and FIG. 2, a testing device for a gas sensor disclosed in the embodiment of the present disclosure includes one thermostatic bath 1 and a gas testing chamber 3 arranged above the thermostatic bath. A humidity tank 4 and a sample tank 11 are provided in the gas testing chamber 3 side by side. The humidity tank 4 is communicated with the sample tank 11 through a gas circuit. The humidity tank 4 and the sample tank 11 extend into the thermostatic bath 1 so that the temperature of the humidity tank 4 is consistent with that of the sample tank 11. What needs illustration is that the humidity tank 4 and the sample tank 11 are located in the same thermostatic bath, so temperature consistency here generally refers to that the temperature inside the humidity tank 4 is consistent with that inside the sample tank 11.

As another embodiment, the difference between the embodiment and the previous embodiment lies in that two thermostatic baths 1 are arranged. The humidity tank 4 and the sample tank 11 are arranged in the different thermostatic baths 1 respectively. At this time, the temperature inside the humidity tank 4 is consistent with that inside the sample tank 11, representing that the temperature difference between the humidity tank 4 and the sample tank 11 is within a preset range (such as 0.1° C. to 0.3° C.) constantly, and slight fluctuation of the temperature difference may exist as long as the humid gas cannot be condensed with such temperature difference.

In the embodiment, the humidity tank 4 and the sample tank 11 are simultaneously arranged in the same thermostatic bath 1. Or, the humidity tank 4 and the sample tank 11 are arranged in the different thermostatic baths 1, but the temperature inside the humidity tank 4 is consistent with that inside the sample tank 11. In the process that the humid gas enters into the sample tank 11, temperature difference does not exist in the gas circuit, so the humid gas cannot be condensed, and then the accuracy of gas humidity is ensured to improve the testing precision of the sensor.

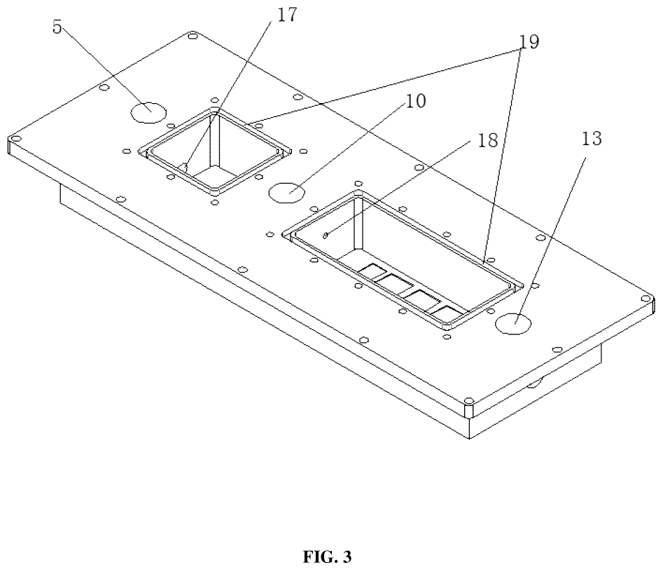

Referring to FIG. 1 and FIG. 3, in one embodiment, a first air inlet passage 5, a second air inlet passage 10 and an air outlet passage 13 are provided in the gas testing chamber 3, the first air inlet passage 5 is communicated with a first air inlet port 17 of the humidity tank 4. The second air inlet passage 10, a first air outlet port of the humidity tank 4 and a second air inlet port of the sample tank 11 jointly form a three-way structure. What needs illustration is that the three-way structure here can be a three-way structure formed by three gas circuits which are perpendicular to each other, and can also be an irregularly three-way structure formed by three gas circuits distributed arbitrarily. The air outlet passage 13 is communicated with the second air outlet port 18 of the sample tank 11, where the first air inlet port 17 is located lower than liquid level inside the humidity tank 4, and the first air outlet port is located higher than the liquid level inside the humidity tank 4, so that it is ensured that gas entering from the first air inlet port 17 is in sufficient contact with liquid inside the humidity tank 4, and the liquid inside the humidity tank 4 can also be prevented from flowing into the first air outlet port.

In a preferable embodiment, the first air inlet passage 5, the second air inlet passage 10 and the air outlet passage 13 are communicated with external gas passages through connecting clamping sleeves 7, respectively.

When gas with certain humidity needs to be introduced, a gas is introduced into the first air inlet port 17 from the first air inlet passage 5. The first air inlet port 17 is lower than liquid level inside the humidity tank 4, so the gas can be in sufficient contact with water to ensure the humidity of gas. A gas introduced from the second air inlet passage 10 communicated with the first air outlet port and the second air inlet port is mixed with the gas with certain humidity which enters into the humidity tank 4 from the first air inlet port 17, and then enters into the sample tank 11 so as to ensure the humidity of gas. Moreover, the humidity of gas can be adjusted by adjusting the quantity of gas in the first air inlet passage 5 and the quantity of gas in the second air inlet passage 10.

When dry gas needs to be introduced, the first air inlet passage 5 or the first air outlet port is plugged, so that the dry gas is directly introduced into the sample tank 11 through the second air inlet passage 10.

Referring to FIG. 1, as a preferable way, the thermostatic bath 1 is a liquid thermostatic bath. A liquid inlet 2 and a liquid outlet 14 are provided in the liquid thermostatic bath. The liquid inlet 2 and the liquid outlet 14 are communicated with a water bath for adjusting a liquid temperature inside the liquid thermostatic bath. In a preferable way, the liquid inlet 2 and the liquid outlet 14 are communicated with the water bath through pagoda-shape connectors 16. The temperature value of the liquid inside the thermostatic bath is adjusted by the water bath, so that the liquid inside the thermostatic bath can be warmed or cooled, and the liquid can be cooled to below zero centigrade avoiding the inability to cool the liquid below zero centigrade when using electric heating wires, which limits the working temperature environment of the sensor. Moreover, the liquid thermostatic bath is directly communicated with the water bath to avoid the problem that the complexity of the device is increased since the humidity is adjusted by the water bath and the device needs to be disassembled and assembled repeatedly.

As a preferable way, the height of the liquid inlet 2 is higher than that of the liquid outlet 14, and the liquid outlet 14 can also be provided at the bottom of the liquid thermostatic bath, so that the accumulation of impurities inside the liquid thermostatic bath is avoided.

The water bath adjusts the temperature of the liquid inside the water bath through a heat pump system provided with a compressor and a heater to realize the warming and cooling of the liquid.

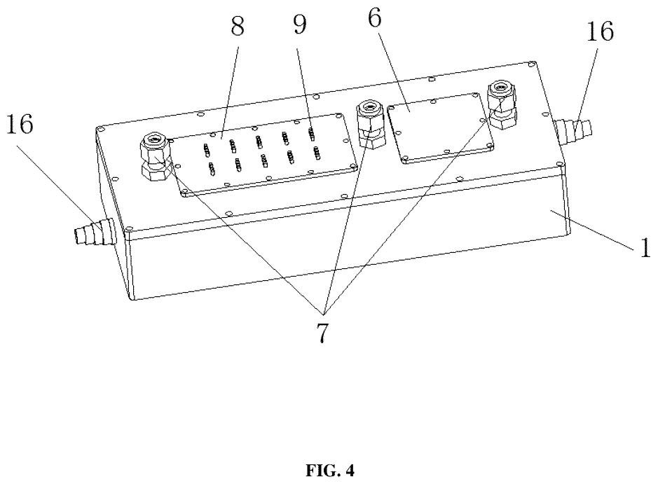

Referring to FIG. 1 and FIG. 4, in a preferable way, a humidity tank cover plate 6 for sealing the humidity tank is arranged above the humidity tank 4, and a sample tank cover plate for sealing the sample tank 11 is arranged above the sample tank 11.

Referring to FIG. 1 and FIG. 2, in a preferable way, the humidity tank cover plate 6 is clamped with the humidity tank 4, and the sample tank cover plate is clamped with the sample tank 11. Through the connection way of clamping, the convenience in the using process can be ensured.

Referring to FIG. 1 and FIG. 4, in a preferred way, the humidity tank cover plate 6 and the sample tank cover plate are connected with the gas testing chamber 3 through threads or fastened with the gas testing chamber 3 through bolts, so that the stability of connection between the humidity tank cover plate 6 and the sample tank cover plate can be ensured.

Referring to FIG. 1, in a preferable way, seal ring grooves 19 for accommodating seal rings 15 are respectively arranged above the humidity tank 4, the sample tank 11 and the thermostatic bath 1, and the seal rings 15 abut against the humidity tank cover plate 6, the sample tank cover plate and the gas testing chamber 3, respectively. The sealing performance of the humidity tank 4, the sample tank 11 and the thermostatic bath 1 can be ensured through the arrangement of the seal rings 15. What needs illustration is that the thermostatic bath 1 and the gas testing chamber can be clamped, connected through screws or fastened through bolts or arranged in other matched manners.

Referring to FIG. 1 and FIG. 3, as a preferable way, the sample tank cover plate is a circuit board 8, and a lower surface of the circuit board 8 is provided with a probe structure 9 for abutting against the gas sensor so as to realize electric connection with the gas sensor.

Preferably, the circuit board 8 is a PCB (Printed Circuit Board) board, and the probe structure 9 is a four-probe testing structure.

Referring to FIG. 1, in a preferable way, multiple grooves 12 are formed in the sample tank 11, and each of the multiple grooves is configured for accommodating the gas sensor. The lower surface of the circuit board 8 is provided with multiple probe structures 9, and the number the grooves 12 is equal to that of the probe structures 9. When the gas sensor needs to be tested, the gas sensor is put in the groove 12, and the circuit board 8 is in sealing connection with the sample tank 11. At this time, the gas sensor is abutted against the probe structure 9 and limits the probe structure 9 to complete the electric connection between the gas sensor and the probe structure 9, so that the error of artificial operation can be reduced, the raw materials can be saved, the testing precision is ensured, and a signal line is prevented from being drawn out artificially on the surface of the gas sensor. In the way of artificially drawing out a signal line, the signal line is in welded connection with a surface of the signal line, inevitably resulting in artificial errors. Therefore, the testing precision is reduced, and the connecting line cannot be recycled after being welded.

Referring to FIG. 1, in a preferred way, the length of the probe structure 9 is greater than the sum of the depth of the sample tank 11 and the depth of the groove 12. The length of the probe structure 9 is greater than the sum of the depth of the sample tank 11 and the depth of the groove 12, so the stability of electric connection between the gas sensor and the probe structure 9 can be ensured, and the connection between the gas sensor and the probe structure 9 is prevented from being untight to cause the condition that the testing precision of the gas sensor is reduced. What needs to be pointed out is that when the circuit board 8 is pressed in the upper part of the sample tank 11, the circuit board 8 can abut against the seal ring 15. The abutting force between the circuit board 8 and the seal ring 15 is adjusted, so that the sealing performance of the sample tank 11 can be ensured, it is ensured that the gas sensor cannot be squashed by the probe structure 9, the stability of electric connection between the gas sensor and the probe structure 9 is ensured, and the problem that the gas sensor is squashed is also avoided.

A using method of the testing device is as follows. When the gas sensor needs to be tested, the gas sensor is put in the groove 12 of the sample tank, and the humidity tank 4 and the sample tank 11 are sealed through the humidity tank cover plate 6 and the PCB board, respectively. At this time, the probe structure 9 abuts against the gas sensor, and the stable electric connection between the gas sensor and the probe structure 9 can be realized. And then, the sample tank 11 and the humidity tank 4 are immersed into the thermostatic bath 1 simultaneously. The gas entering into the humidity tank 4 through the first air inlet passage 5 is mixed with the gas entering into the second air inlet passage 10. After the humidity is adjusted, the gas mixture enters into the sample tank 11. At this time, temperature difference does not exist between the sample tank 11 and the humidity tank 4, and the humid gas cannot be condensed in the sample tank 11 or the pipeline, so the accuracy of gas humidity is ensured. And then, the testing of the gas sensor is completed by contrasting the temperature and humidity of the gas tested by the gas sensor with the original temperature and humidity of the gas.

The present disclosure is not limited to details in the above-mentioned exemplary embodiments. All other embodiments obtained by those skilled in the art based on the embodiments of the present disclosure without creative efforts shall fall within the protection scope of the present disclosure.

What is claimed is:

1. A testing device for a gas sensor, comprising a thermostatic bath and a gas testing chamber arranged above the thermostatic bath, wherein a humidity tank for adjusting gas humidity and a sample tank for testing the gas sensor are provided in the gas testing chamber, the sample tank is communicated with the humidity tank through a gas circuit, and the humidity tank and the sample tank extend into the thermostatic bath, so that a temperature of the humidity tank is consistent with that of the sample tank.

2. The testing device for a gas sensor according to claim 1, wherein a first air inlet passage, a second air inlet passage and an air outlet passage are provided in the gas testing chamber, the first air inlet passage is communicated with a first air inlet port of the humidity tank, the second air inlet passage, a first air outlet port of the humidity tank and a second air inlet port of the sample tank jointly form a three-way structure, and the air outlet passage is communicated with a second air outlet port of the sample tank, wherein the first air inlet port is located lower than liquid level inside the humidity tank, and the first air outlet port is located higher than the liquid level inside the humidity tank.

3. The testing device for a gas sensor according to claim 1, wherein the thermostatic bath is a liquid thermostatic bath, a liquid inlet and a liquid outlet are provided in the liquid thermostatic bath, and the liquid inlet and the liquid outlet are communicated with a water bath for adjusting a liquid temperature inside the liquid thermostatic bath.

4. The testing device for a gas sensor according to claim 1, wherein a humidity tank cover plate for sealing the humidity tank is arranged above the humidity tank, and a sample tank cover plate for sealing the sample tank is arranged above the sample tank.

5. The testing device for a gas sensor according to claim 4, wherein the humidity tank cover plate is clamped with the humidity tank, and the sample tank cover plate is clamped with the sample tank.

6. The testing device for a gas sensor according to claim 4, wherein the humidity tank cover plate and the sample tank cover plate are connected with the gas testing chamber through threads.

7. The testing device for a gas sensor according to claim 4, wherein seal ring grooves for accommodating seal rings are respectively arranged above the humidity tank and the sample tank, and the seal rings abut against the humidity tank cover plate and the sample tank cover plate, respectively.

8. The testing device for a gas sensor according to claim 4, wherein the sample tank cover plate is a circuit board, and a lower surface of the circuit board is provided with a probe structure for abutting against the gas sensor so as to realize electric connection with the gas sensor.

9. The testing device for a gas sensor according to claim 8, wherein a plurality of grooves for are formed in the sample tank, each of the plurality of grooves is configured for accommodating the gas sensor, the lower surface of the circuit board is provided with a plurality of probe structures, and a number of the grooves is equal to that of the plurality of probe structures.

10. The testing device for a gas sensor according to claim 9, wherein a length of each of the plurality of probe structures is greater than a sum of a depth of the sample tank and a depth of each of the plurality of grooves.
